(19) United States
(12) Patent Application Publication
(10) Pub. No.: US 2020/0405266 A1
(43) Pub. Date: Dec. 31, 2020

(54) METHODS AND APPARATUSES FOR PROCESSING ULTRASOUND SIGNALS

(71) Applicants: Jungwook Yang, Newton, MA (US); Daniel Rea McMahill, Woodstock, GA (US); Nevada J. Sanchez, Guilford, CT (US)

(72) Inventors: Jungwook Yang, Newton, MA (US); Daniel Rea McMahill, Woodstock, GA (US); Nevada J. Sanchez, Guilford, CT (US)

(73) Assignee: Butterfly Network, Inc., Guilford, CT (US)

(21) Appl. No.: 16/910,657

(22) Filed: Jun. 24, 2020

Related U.S. Application Data
(60) Provisional application No. 62/866,198, filed on Jun. 25, 2019.

(51) Int. Cl. A61B 8/08; A61B 8/00; A61B 8/06; G01S 15/89; G01S 7/52

(52) U.S. Cl. CPC A61B 8/5207; A61B 8/4483; A61B 8/488; A61B 8/06; G01S 7/5208; G01S 15/8965; G01S 15/8979; G01S 15/8915; G01S 7/52025; A61B 8/56

(57) ABSTRACT

Aspects of the technology described herein relate to a pipeline configured to pipeline ultrasound signals from multiple analog front-ends (AFEs) to a digital portion of an ultrasound processing unit. The ultrasound signals may be digital ultrasound signals from analog-to-digital converters of the multiple AFEs. The pipeline may include first pipelining circuitry in a first AFE and second pipelining circuitry in a second AFE. The first pipelining circuitry may be configured to output a first digital ultrasound signal from the first pipelining circuitry to the digital portion of the UPU, receive a second digital ultrasound signal from second pipelining circuitry, and output the second digital ultrasound signal from the first pipelining circuitry to the digital portion of the UPU. De-interleaving circuitry may be coupled to the first pipelining circuitry and configured to de-interleave the first digital ultrasound signal and the second digital ultrasound signal outputted by the first pipelining circuitry.

### CROSS-REFERENCE TO RELATED APPLICATIONS

The present application claims the benefit under 35 U.S.C. § 119(e) of U.S. Patent Application Ser. No. 62/866,198, filed Jun. 25, 2019 under Attorney Docket No. B1348.70146US00 and entitled “METHODS AND APPARATUSES FOR PROCESSING ULTRASOUND SIGNALS,” which is hereby incorporated herein by reference in its entirety.

### FIELD

Generally, the aspects of the technology described herein relate to processing ultrasound signals. Some aspects relate to methods and apparatuses for pipelining ultrasound signals.

### BACKGROUND

Ultrasound devices may be used to perform diagnostic imaging and/or treatment, using sound waves with frequencies that are higher than those audible to humans. Ultrasound imaging may be used to see internal soft tissue body structures. When pulses of ultrasound are transmitted into tissue, sound waves of different amplitudes may be reflected back towards the probe at different tissue interfaces. These reflected sound waves may then be recorded and displayed as an image to the operator. The strength (amplitude) of the sound signal and the time it takes for the wave to travel through the body may provide information used to produce the ultrasound image. Many different types of images can be formed using ultrasound devices. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect, an ultrasound processing unit (UPU) comprises a pipeline configured to pipeline ultrasound signals from multiple analog front-ends (AFEs) to a digital portion of the UPU.

In some embodiments, the ultrasound signals are digital ultrasound signals from analog-to-digital converters (ADCs) of the multiple AFEs. In some embodiments, the pipeline includes first pipelining circuitry in a first AFE of the multiple AFEs and second pipelining circuitry in a second AFE of the multiple AFEs; and the first pipelining circuitry is configured to: output a first digital ultrasound signal from the first pipelining circuitry to the digital portion of the UPU; receive a second digital ultrasound signal from second pipelining circuitry; and output the second digital ultrasound signal from the first pipelining circuitry to the digital portion of the UPU.

In some embodiments, the first pipelining circuitry is configured to output the first digital ultrasound signal and the second digital ultrasound signal to the digital portion of the UPU in an interleaved fashion. In some embodiments, the UPU further includes de-interleaving circuitry coupled to the first pipelining circuitry and configured to de-interleave the first digital ultrasound signal and the second digital ultrasound signal outputted by the first pipelining circuitry. In some embodiments, the digital portion includes the de-interleaving circuitry. In some embodiments, the digital portion includes digital processing circuitry.

In some embodiments, the first AFE includes an analog-to-digital converter (ADC) coupled to the first pipelining circuitry and configured to convert a first analog ultrasound signal to the first digital ultrasound signal; the second AFE includes an ADC coupled to the second pipelining circuitry and configured to convert a second analog ultrasound signal to the second digital ultrasound signal; the UPU further includes a data bus extending from the second pipelining circuitry to the first pipelining circuitry; and the first pipelining circuitry is configured to receive the second digital ultrasound signal from the second pipelining circuitry over the data bus. In some embodiments, the first AFE is disposed between the second AFE and the digital portion of the UPU.

In some embodiments, the first AFE further includes a pulser, a switch, and analog processing circuitry. In some embodiments, an ultrasound-on-chip includes the UPU, and the first and second AFEs are arranged along an elevational dimension of the ultrasound-on-chip. In some embodiments, the UPU further includes ultrasonic transducers physically located on top of each of the first and second AFEs and arranged along the elevational dimension of the ultrasound-on-chip. In some embodiments, the ultrasound-on-chip includes an array of ultrasonic transducers along an azimuthal dimension and an elevational dimension of the ultrasound-on-chip. In some embodiments, each of the first and second digital ultrasound signals includes a number of bits, and a number of wires carrying digital ultrasound signals from ADCs of the UPU and passing over the first AFE is equal to the number of bits.

In some embodiments, the pipelining circuitry of the first AFE is configured to: output the first digital ultrasound signal from the first pipelining circuitry to the digital portion of the UPU and to receive the second digital ultrasound signal from the second pipelining circuitry on a first clock ultrasound signal; and output the second digital ultrasound signal from the first pipelining circuitry to the digital portion of the UPU on a second clock ultrasound signal.

In some embodiments, each of the first pipelining circuitry and the second pipelining circuitry includes: a multiplexer including a first data input, a second data input, and a data output; and a flip-flop including a data input and a data output; wherein: the data output of the multiplexer is coupled to the data input of the flip-flop. In some embodiments, the first data input of the multiplexer in the second pipelining circuitry is coupled to an ADC in the second AFE; the data output of the flip-flop in the second pipelining circuitry is coupled to the second data input of the multiplexer of the first pipelining circuitry; the first data input of the multiplexer in the first pipelining circuitry is coupled to an ADC in the first AFE; and the data output of the flip-flop in the first pipelining circuitry is coupled to the digital portion of the UPU. In some embodiments, the de-interleaving circuitry includes a demultiplexer including a first data input and multiple data outputs.

In some embodiments, the first pipelining circuitry includes: a multiplexer including a first data input, a second data input, and a data output; and a flip-flop including a data input and a data output; wherein: the data output of the multiplexer is coupled to the data input of the flip-flop; and the de-interleaving circuitry includes: a demultiplexer including a data input and multiple data outputs; wherein: the data output of the flip-flop of the first pipelining circuitry is coupled to the data input of the demultiplexer; and the multiple data outputs of the demultiplexer are coupled to the digital portion of the UPU. In some embodiments, the second pipelining circuitry includes: a flip-flop including a data input and a data output; the first pipelining circuitry includes: a first flip-flop including a data input and a data output; and a second flip-flop including a data input and a data output; the data input of the flip-flop in the second pipelining circuitry is coupled to an ADC in the second AFE; the data output of the flip-flop in the second pipelining circuitry is coupled to the data input of the second flip-flop in the first pipelining circuitry; the data input of the first flip-flop in the first pipelining circuitry is coupled to an ADC in the first AFE; and the data outputs of the first and second flip-flops in the first pipelining circuitry are coupled to the digital portion of the UPU.

Some aspects include a method to perform the actions that the UPU is configured to perform.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 illustrates an example physical layout of a portion of an ultrasound-on-chip, in accordance with certain embodiments described herein;

FIG. 2 illustrates an example physical layout of an ultrasound processing unit (UPU) in the ultrasound-on-chip of FIG. 1, in accordance with certain embodiments described herein;

FIG. 3 illustrates an example block diagram of circuitry in the ultrasound-on-chip of FIG. 1, in accordance with certain embodiments described herein;

FIG. 4 illustrates an example block diagram of circuitry in the ultrasound-on-chip of FIG. 1, in accordance with certain embodiments described herein;

FIG. 5 illustrates an example block diagram of circuitry in the ultrasound-on-chip of FIG. 1, in accordance with certain embodiments described herein;

FIG. 6 illustrates an example block diagram of circuitry in the ultrasound-on-chip of FIG. 1, in accordance with certain embodiments described herein;

FIG. 7 illustrates example pipelining circuitry, in accordance with certain embodiments described herein;

FIG. 8 illustrates an example pipeline, in accordance with certain embodiments described herein;

FIG. 9 illustrates an example pipeline, in accordance with certain embodiments described herein;

FIG. 10 illustrates an example pipeline, in accordance with certain embodiments described herein;

FIG. 11 illustrates an example pipeline, in accordance with certain embodiments described herein;

FIG. 12 illustrates example de-interleaving circuitry, in accordance with certain embodiments described herein;

FIG. 13 illustrates another example of pipelining circuitry, in accordance with certain embodiments described herein;

FIG. 14 illustrates another example pipeline, in accordance with certain embodiments described herein;

FIG. 15 illustrates an example handheld ultrasound probe, in accordance with certain embodiments described herein;

FIG. 16 illustrates an example wearable ultrasound patch, in accordance with certain embodiments described herein;

FIG. 17 illustrates an example ingestible ultrasound pill, in accordance with certain embodiments described herein; and

FIG. 18 illustrates a process for processing ultrasound signals, in accordance with certain embodiments described herein.

### DETAILED DESCRIPTION

When an analog-to-digital converter (ADC) outputs a new binary value that changes digital values on certain bits of its output data bus, this may cause a draw in current from the power supply, power supply noise, and/or transfer of this digital switching activity through capacitive coupling to nearby analog signals These signals may be high-precision analog signals, and may be low bandwidth and/or low amplitude analog signals. Transfer of digital switching activity through capacitive coupling to nearby analog signals can cause noise in measurements based on the analog signals. In conventional integrated circuits, analog circuitry may be adjacent to an ADC, and the ADC may be adjacent to digital circuitry. Thus, the data bus from the ADC which is routed to the digital circuitry may not need to be routed over the analog circuitry, and so digital switching on the data bus may be protected from generating noise in analog signals in the analog circuitry.

The inventors have developed ultrasound-on-chip technology. Such technology may include integrating a large number of ultrasonic transducers along both the elevational and azimuthal dimensions as well as ultrasound circuitry on a single semiconductor chip or on multiple semiconductor chips arranged in a stacked configuration. Such an ultrasound-on-chip may form the core of a handheld ultrasound probe or an ultrasound device having another form factor such as a wearable ultrasound patch or an ingestible ultrasound pill. The large ultrasound transducer array may allow such ultrasound devices to have advanced functionality in terms of imaging techniques and clinical uses. For further description of an ultrasound-on-chip, see U.S. patent application Ser. No. 15/626,711 titled “UNIVERSAL ULTRASOUND IMAGING DEVICE AND RELATED APPARATUS AND METHODS,” filed on Jun. 19, 2017 and published as U.S. Pat. App. Publication No. 2017-0360399 A1 (and assigned to the assignee of the instant application), which is incorporated by reference herein in its entirety.

Ultrasound-on-chips may include multiple analog front-ends (AFEs) for processing signal from ultrasonic transducers. Some embodiments may include multiple AFEs, each including an analog-to-digital converter, tiled along the elevational dimension of the ultrasound-on-chip. Each AFE may be configured to process signals from ultrasonic transducers at different locations along the elevational dimension of the ultrasound-on-chip. The AFEs may share digital circuitry, and this may require data buses from ADCs in certain AFEs to be routed over analog circuits in nearby AFEs in order to reach the shared digital circuitry. This may increase the possibility of digital switching on the data bus generating noise in analog signals in the nearby AFEs' analog circuitry. If, for example, a UPU has a column of m AFEs and a digital portion, and each AFE outputs an n-bit digital value on a bus of n wires directly to the digital portion, there may be m×n wires passing over the last AFE before they reach the digital portion, and all m×n wires may contribute to generating noise due to digital switching.

The inventors have realized that instead of having data buses from each of the AFEs extending directly to the digital portion of the UPU, pipelining may be used to transmit output data from each of the AFEs to the digital portion. In particular, one of the AFEs may pass its output data to an adjacent AFE, and that AFE may then pass the output data from the previous AFE to an adjacent AFE, and so on, until an AFE passes the output data to the digital portion of the UPU. This may only require each AFE to pass n wires to an adjacent AFE (or to the digital portion of the UPU). Thus, instead of m×n wires from the AFEs all passing over the last AFE to reach the digital portion in the case where each data bus extends directly from each of the AFEs to the digital portion, only n wires (from the last AFE) may pass over the last AFE to reach the digital portion. Thus, the effect of digital switching from data buses on analog circuitry in the AFEs may be reduced. These n wires may transmit a digital signal that interleaves the digital signals from all the AFEs in the pipeline. This interleaved signal may be de-interleaved upon reaching the digital portion of the UPU.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

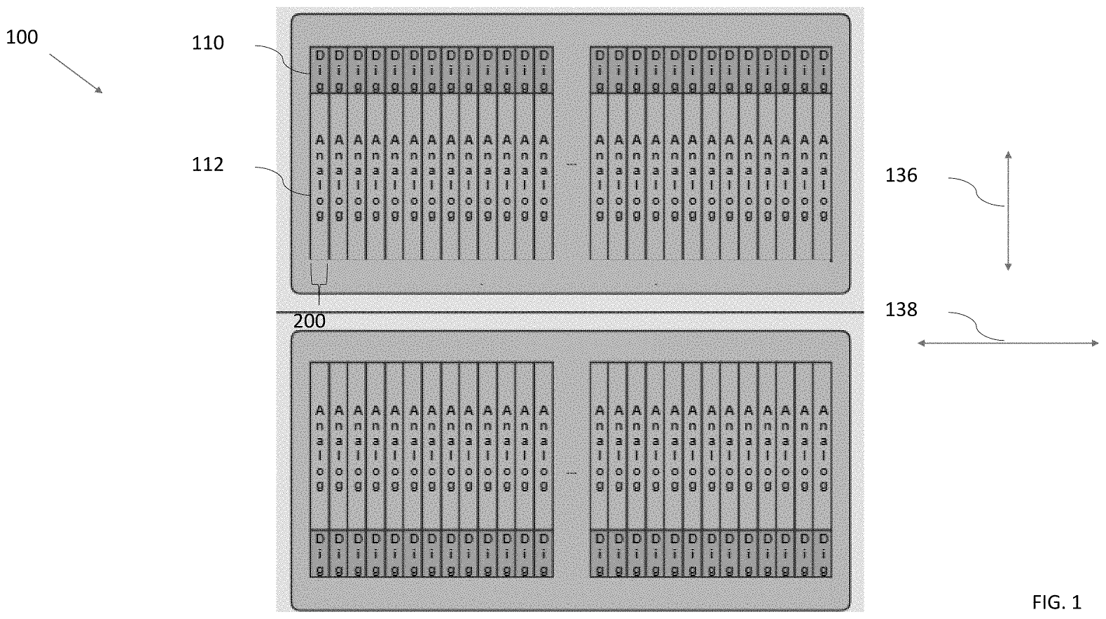

FIG. 1 illustrates an example physical layout of a portion of an ultrasound-on-chip 100, in accordance with certain embodiments described herein. The ultrasound-on-chip 100 is illustrated in FIG. 1 from a bird's-eye-view. FIG. 1 illustrates the ultrasound-on-chip 100, multiple ultrasound processing units (UPUs) 200 in the ultrasound-on-chip 100, an elevational dimension 136 of the ultrasound-on-chip 100, and an azimuthal dimension 138 of the ultrasound-on-chip 100. Each UPU 200 may be a self-contained ultrasound processing unit that forms a sub-array of a complete ultrasound imaging array in a scalable fashion. Each UPU 200 includes an analog portion 112 and a digital portion 110, and may include, for example, any or all of high-voltage pulsers to drive ultrasonic transducers to emit ultrasound; analog and mixed-signal receiver channels to receive and digitize ultrasound echoes; digital processing circuitry to filter, compress, and/or beamform the digital data from each channel; and digital sequencing circuit to control and coordinate different parts of the circuitry to work in sync. The analog portion 112 is physically separated from the digital portion 110. FIG. 1 illustrates how multiple UPUs 200 are tiled along an azimuthal dimension 138 of the ultrasound-on-chip 100, and how two rows of the tiled UPUs 200 are arranged along the elevational dimension 136 of the ultrasound-on-chip 100. Ultrasonic transducers (not shown in FIG. 1) may be physically located on top of (i.e., with respect to the depth dimension of the ultrasound-on-chip 100, out of the plane of FIG. 1) the analog portion 112 of each UPU 200, along the elevational dimension 136 of the ultrasound-on-chip 100. Due to the arrangement of the ultrasonic transducers on top of the analog portion 112 of each UPU 200, along the elevational dimension 136 of the ultrasound-on-chip 100, and due to the tiling of the UPUs 200 along the azimuthal dimension 138 of the ultrasound-on-chip 100, ultrasonic transducers may be arranged in an array along the azimuthal dimension 138 and elevational dimensions 136 of the ultrasound-on-chip 100, thus allowing for azimuthal and elevational beamforming of ultrasound signals received by the ultrasound-on-chip 100.

The physical layout of the ultrasound-on-chip 100 as illustrated in FIG. 1 is non-limiting. For example, in some embodiments, the ultrasound-on-chip 100 may have fewer UPUs 200 than shown, more or fewer UPUs 200 in each row than shown, and/or more or fewer rows of UPUs 200 than shown. Additionally, while in FIG. 1, the digital portion 110 of each UPU 200 is at the edge of the ultrasound-on-chip 100 and the analog portion 112 of each UPU 200 is at the center of the ultrasound-on-chip 100, in some embodiments the digital portion 110 of each or some of the UPUs 200 may be at the center of the ultrasound-on-chip 100 and the analog portion 112 of each or some UPUs 200 may be at the edge of the ultrasound-on-chip 100.

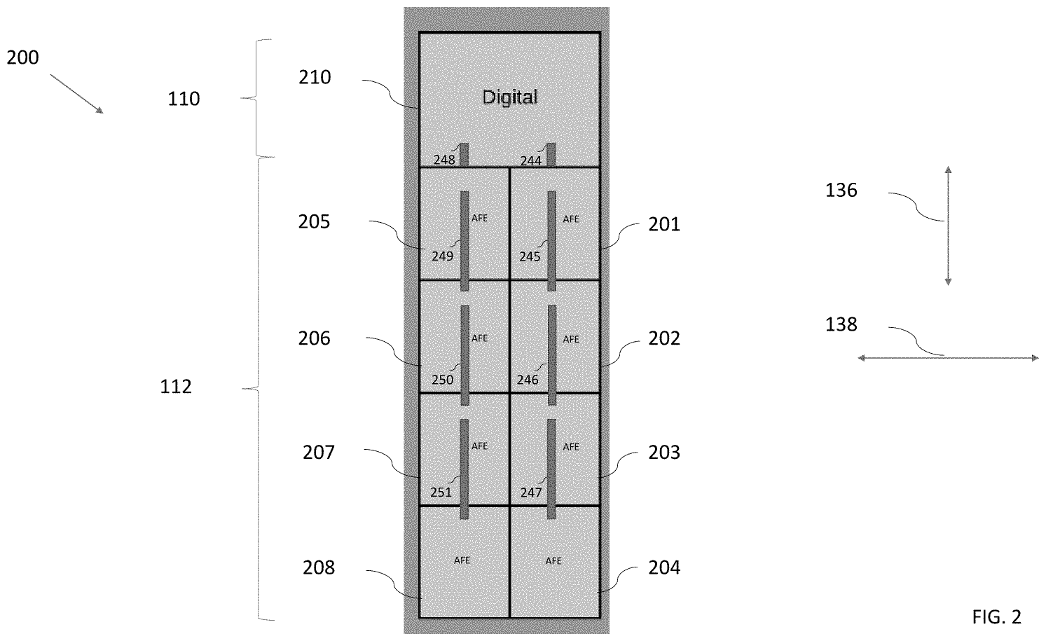

FIG. 2 illustrates an example physical layout of an ultrasound processing unit (UPU) 200 of the ultrasound-on-chip 100 of FIG. 1, in accordance with certain embodiments described herein. The UPU 200 is illustrated in FIG. 2 from a bird's-eye-view of the ultrasound-on-chip 100. The UPU 200 includes eight analog front-ends (AFE) 201-208, a block of digital circuitry 210, and eight data buses 244-251. The AFEs 201-208 are part of the analog portion 112 of the UPU 200 and the digital circuitry 210 is part of the digital portion 110 of the UPU 200. The AFEs 201-208 are physically arranged in two columns, with each of the columns including four AFEs arranged along the elevational dimension 136 of the ultrasound-on-chip 100. The digital circuitry 210 is physically located at one end of the columns along the elevational dimension 136 of the ultrasound-on-chip 100. As will be described in further detail below, each of the AFEs 201-208 may include a pulser, a switch, analog processing circuitry, an ADC, and pipelining circuitry. As described above, ultrasonic transducers (not shown in FIG. 2) may be physically located on top of (i.e., with respect to the depth dimension of the ultrasound-on-chip 100, out of the plane of FIG. 2) each of the AFEs 201-208. For example, multiple ultrasonic transducers (e.g., eight) may be located each of the AFEs 201-208, and each of the transducers may be coupled to the circuitry of the respective AFE in a multiplexed fashion. Thus, ultrasonic transducers may be arranged on top of each UPU 200, along the elevational dimension 136 of the ultrasound-on-chip 100.

As will also be described in detail below, the digital circuitry 210 may include a waveform generator, de-interleaving circuitry, and digital processing circuitry. The digital circuitry 210 may be configured to process signals from the AFEs 201-208. Thus, output signals from each of the AFEs 201-208 (e.g., output signals from ADCs) may be routed to the digital circuitry 210, which may process the signals from each of the AFEs 201-208 in a multiplexed fashion. As can be seen in FIG. 2, due to the physical layout of the UPU 200, and specifically the arrangement of AFEs 201-208 along the elevational dimension 136 of the ultrasound-on-chip 100, if a data bus were to extend directly from certain of the AFEs 201-208 to the digital circuitry 210, that data bus may pass over other of the AFEs 201-208 to reach the digital circuitry 210. For example, consider that each of the AFEs 201-208 outputs an n-bit value on a data bus having n wires, one wire for each bit. The data buses from, for example, the AFEs 206-208 may all need to pass over the AFE 205 to reach the digital circuitry 210, if each data bus extended directly from each of the AFEs 206-208 to the digital circuitry 210. Thus, 4n wires (n wires from the AFE 205 and 3n wires from the AFEs 206-208) may all need to pass over the AFE 205 to reach the digital circuitry 210. Digital switching activity on all 4n wires may cause noise in the analog circuitry of the AFE 205. It should be appreciated that four AFEs in a column is an example, and in embodiments with m AFEs in a column in a UPU, there may be m×n wires passing over the last AFE closest to the digital circuitry 210.

The inventors have recognized that instead of data buses from each of the AFEs 201-208 extending directly to the digital circuitry 210, pipelining may be used to transmit output data from each of the AFEs 201-208 to the digital circuitry 210. In particular, one of the AFEs may pass its output data to an adjacent AFE, and that AFE may pass the output data from the previous AFE to an adjacent AFE, and so on, until an AFE passes the output data to the digital circuitry 210. Thus, as FIG. 2 illustrates, the data bus 244 passes from the AFE 201 to the digital circuitry 210, the data bus 245 passes from the AFE 202 to the AFE 201, the data bus 246 passes from the AFE 203 to the AFE 202, the data bus 247 passes from the AFE 204 to the AFE 203, the data bus 248 passes from the AFE 205 to the digital circuitry 210, the data bus 249 passes from the AFE 206 to the AFE 205, the data bus 250 passes from the AFE 207 to the AFE 206, and the data bus 251 passes from the AFE 208 to the AFE 207. Continuing the example described above, it should be appreciated that instead of 4n wires from the AFEs 206-208 all passing over the AFE 205 to reach the digital circuitry 210 in the case where each data bus extends directly from each of the AFEs 206-208 to the digital circuitry 210, only n wires (from the AFE 205) may pass over the AFE 205 to reach the digital circuitry 210. Thus, the effect of digital switching from data buses on analog circuitry in the AFE 205 may be reduced. It should be appreciated that the pipelining scheme of FIG. 2 may also reduce the number of wires passing over the AFEs 206-207.

As an example of operation of the pipeline, on one clock cycle, the AFE 208 may pass its output data to the AFE 207 over the data bus 251, the AFE 207 may pass its output data to the AFE 206 over the data bus 250, the AFE 206 may pass its output data to the AFE 205 over the data bus 249, and the AFE 205 may pass its output data to the digital circuitry 210 over the data bus 248. On the next clock cycle, the AFE 207 may pass the AFE 208's output data to the AFE 206 over the data bus 250, the AFE 206 may pass the AFE 207's output data to the AFE 205 over the data bus 249, and the AFE 205 may pass the AFE 206's output data to the digital circuitry 210 over the data bus 248. On the next clock cycle, the AFE 206 may pass the AFE 208's output data to the AFE 205 over the data bus 249, and the AFE 205 may pass the AFE 207's output data to the digital circuitry 210 over the data bus 248. On the next clock cycle, the AFE 205 may pass the AFE 208's output data to the digital circuitry 210 over the data bus 248. When each of the AFEs 205-208 has generated new output data, the process may repeat.

The physical layout of the UPU 200 as illustrated in FIG. 2 is non-limiting. For example, in some embodiments, there may be more or fewer AFEs than shown, more or fewer AFEs per column than shown, and/or more or fewer columns than shown. Additionally, in some embodiments, the data buses may take different paths from the AFEs to the digital circuitry 210 than shown.

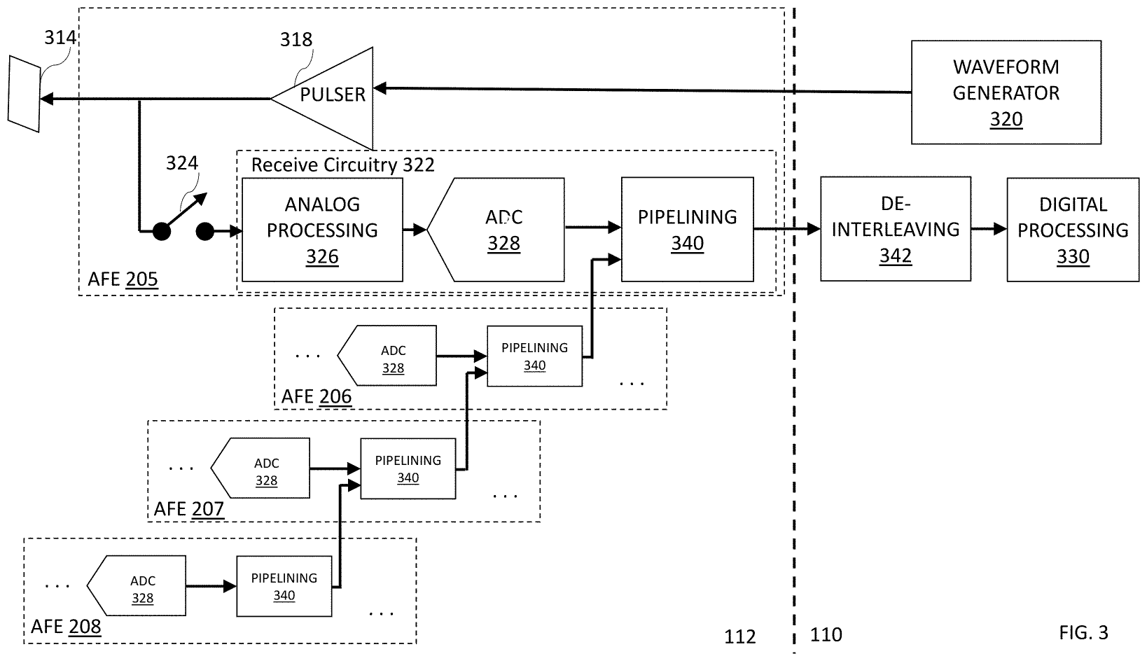

FIG. 3 illustrates an example block diagram of circuitry in the ultrasound-on-chip 100, in accordance with certain embodiments described herein. The circuitry illustrated in FIG. 3 should be understood to be only a portion of the circuitry in the ultrasound-on-a-chip 100. FIG. 3 illustrates in detail exemplary circuitry that may be included in the AFEs 205-208. More specifically, the exemplary circuitry in the AFE 205 includes a pulser 318, a waveform generator 320, a switch 324, analog processing circuitry 326, an analog-to-digital converter (ADC) 328, and pipelining circuitry 340. The analog processing circuitry 326, the ADC 328, and the pipelining circuitry 340 constitute receive circuitry 322. The circuitry in the AFEs 206-208 includes the same circuitry as in the AFE 205, but for simplicity, only the ADC 328 and the pipelining circuitry 340 are illustrated for each. The circuitry illustrated in FIG. 3 further includes an ultrasonic transducer 314, de-interleaving circuitry 342, and digital processing circuitry 330. As illustrated, the ultrasonic transducers 314, the pulsers 318, the switches 324, the blocks of analog processing circuitry 326, the ADCs 328, and the blocks of pipelining circuitry 340 are in the analog portion 112 of the UPU 200. The waveform generator 320, the de-interleaving circuitry 342, and the digital processing circuitry 330 are in the digital portion 110 of the UPU 200.

The waveform generator 320 may be configured to provide a waveform to the pulser 318. The pulser 318 may be configured to output a driving signal corresponding to the received waveform to the ultrasonic transducer 314. When the pulser 318 is driving the ultrasonic transducer 314 (the “transmit phase”), the switch 324 may be open such that the driving signal is not applied to the receive circuitry 322.

The ultrasonic transducer 314 may be configured to emit pulsed ultrasonic signals into a subject, such as a patient, in response to the driving signal received from the pulser 318. The pulsed ultrasonic signals may be back-scattered from structures in the body, such as blood cells or muscular tissue, to produce echoes that return to the ultrasonic transducer 314. The ultrasonic transducer 314 may be configured to convert these echoes into electrical signals (i.e., analog ultrasound signals). When the ultrasonic transducer 314 is receiving the echoes (the “receive phase”), the switch 324 may be closed such that the ultrasonic transducer 314 may transmit the analog ultrasound signals representing the received echoes through the switch 324 to the receive circuitry 322.

The analog processing circuitry 326 may include, for example, one or more analog amplifiers, one or more analog filters, analog beamforming circuitry, analog dechirp circuitry, analog quadrature demodulation (AQDM) circuitry, analog time delay circuitry, analog phase shifter circuitry, analog summing circuitry, analog time gain compensation circuitry, and/or analog averaging circuitry. The analog ultrasound signal output of the analog processing circuitry 326 is outputted to the ADC 328 for conversion to a digital signal. The ADC 328 may be, for example, a flash ADC, a successive approximation ADC, or a sigma-delta ADC configured to convert analog signals to digital signals. The digital ultrasound signal output of the ADC 328 is outputted to the pipelining circuitry 340.

The pipelining circuitry 340 of the AFEs 205-208 may be configured to pipeline data from the AFEs 205-208 (in particular, from the ADCs 328 of the AFEs 205-208) to the digital portion 110 of the UPU 200 (in particular, to the de-interleaving circuitry 342). The pipeline includes a chain of pipelining circuitry 340 in each of the AFEs 205-208 passing their outputs (e.g., outputs of the ADCs 328) from one AFE to another until they reach the digital portion 110 of the UPU 200. Data from the ADC 328 of the AFE 208 may pass through the pipelining circuitry 340 of the AFEs 208-205 before reaching the de-interleaving circuitry 342, data from the ADC 328 of the AFE 207 may pass through the pipelining circuitry 340 of the AFEs 207-205 before reaching the de-interleaving circuitry 342, data from the ADC 328 of the AFE 206 may pass through the pipelining circuitry 340 of the AFEs 206-205 before reaching the de-interleaving circuitry 342, and data from the ADC 328 of the AFE 205 may pass through the pipelining circuitry 340 of the AFE 205 before reaching the de-interleaving circuitry 342. Data from the ADC 328 of the AFE 205 may reach the de-interleaving circuitry 342 first, followed by data from the ADC 328 of the AFE 206, followed by data from the ADC 328 of the AFE 207, followed by data from the ADC 328 of the AFE 208, etc. Thus, the signal provided at the input of the de-interleaving circuitry 342 may be an interleaved signal including data from the ADC 328 of the AFE 208, data from the ADC 328 of the AFE 207, data from the ADC 328 of the AFE 206, and data from the ADC 328 of the AFE 205.

The de-interleaving circuitry 342 may be configured to de-interleave the interleaved signal that is provided at its input, which may include data from the ADC 328 of the AFE 208, data from the ADC 328 of the AFE 207, data from the ADC 328 of the AFE 206, and data from the ADC 328 of the AFE 205 interleaved with each other. In particular, the de-interleaving circuitry 342 may be configured to split the interleaved signal at its output into separate signals, one for data from the ADC 328 of the AFE 208, one for data from the ADC 328 of the AFE 207, one for data from the ADC 328 of the AFE 206, and one for data from the ADC 328 of the AFE 205.

The de-interleaved signals from the de-interleaving circuitry 342 may be outputted to the digital processing circuitry 330. The digital processing circuitry 330 may include, for example, one or more digital filters, digital beamforming circuitry, digital quadrature demodulation (DQDM) circuitry, averaging circuitry, digital dechirp circuitry, digital time delay circuitry, digital phase shifter circuitry, digital summing circuitry, digital multiplying circuitry, requantization circuitry, waveform removal circuitry, image formation circuitry, and backend processing circuitry. The image formation circuitry may be configured to perform apodization, back projection and/or fast hierarchy back projection, interpolation range migration (e.g., Stolt interpolation) or other Fourier resampling techniques, dynamic focusing techniques, and/or delay and sum techniques, tomographic reconstruction techniques, etc.

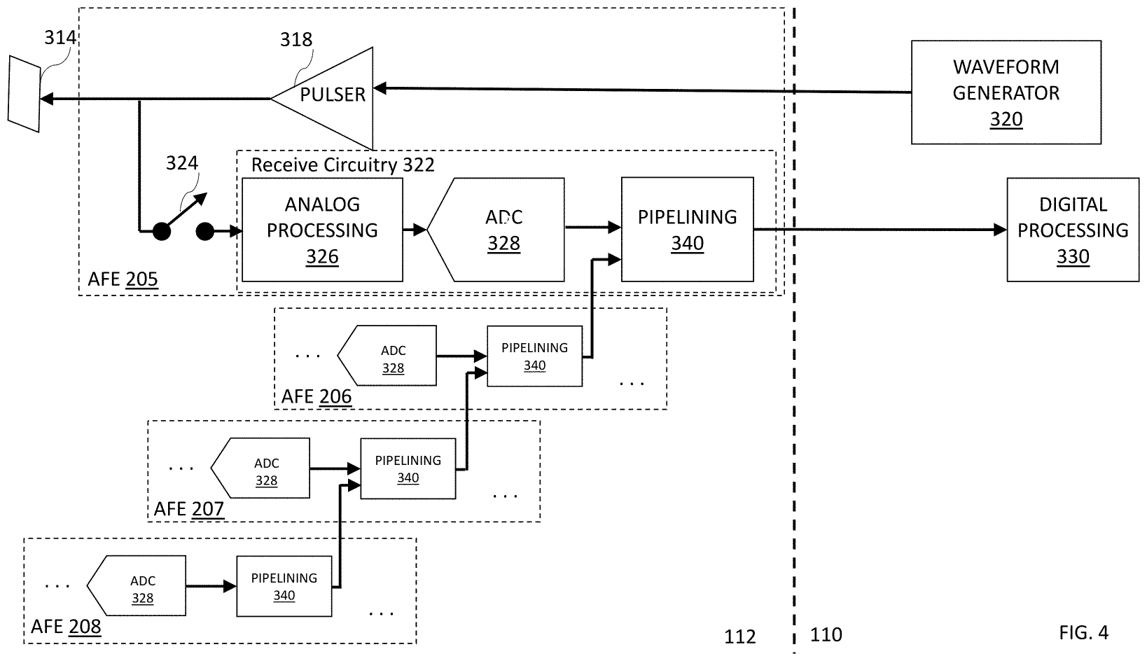

FIG. 4 illustrates another example block diagram of circuitry in the ultrasound-on-chip 100, in accordance with certain embodiments described herein. FIG. 4 differs from FIG. 3 in that the embodiment of FIG. 4 lacks the de-interleaving circuitry 342. In this embodiment, the digital processing circuitry 330 may be configured to digitally process interleaved signals.

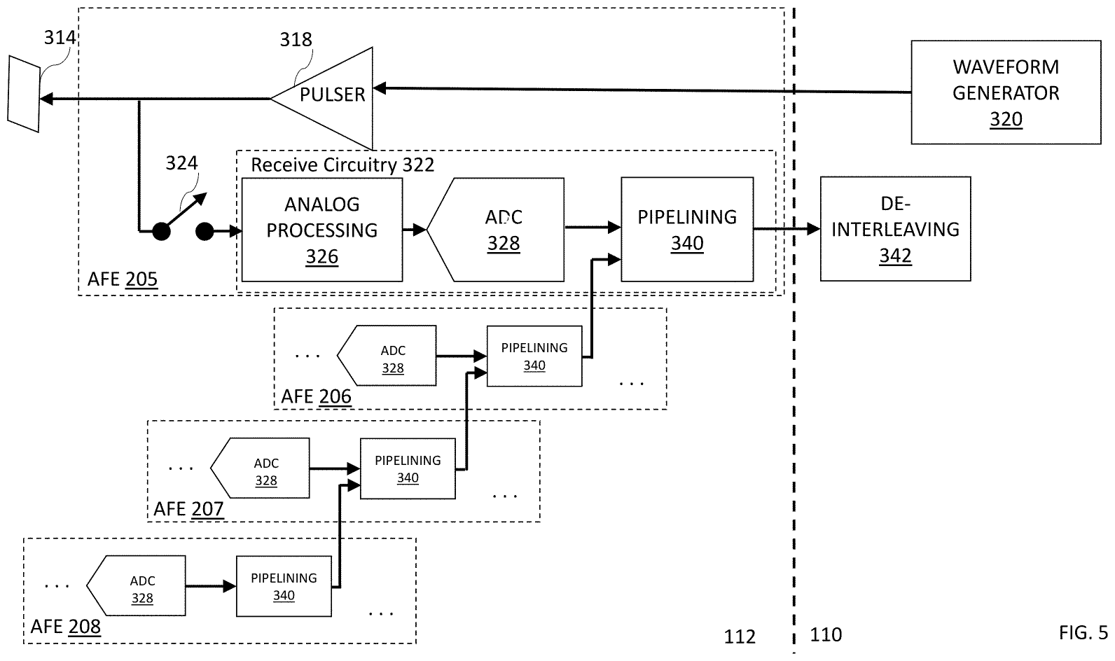

FIG. 5 illustrates another example block diagram of circuitry in the ultrasound-on-chip 100, in accordance with certain embodiments described herein. FIG. 5 differs from both FIG. 3 and FIG. 4 in that the embodiment of FIG. 5 lacks the digital processing circuitry 330. In this embodiment, the ultrasound-on-a-chip 100 may be configured to output the de-interleaved signals to an off-chip device (e.g., a field-programmable gate array (FPGA)), which may be configured to perform digital processing.

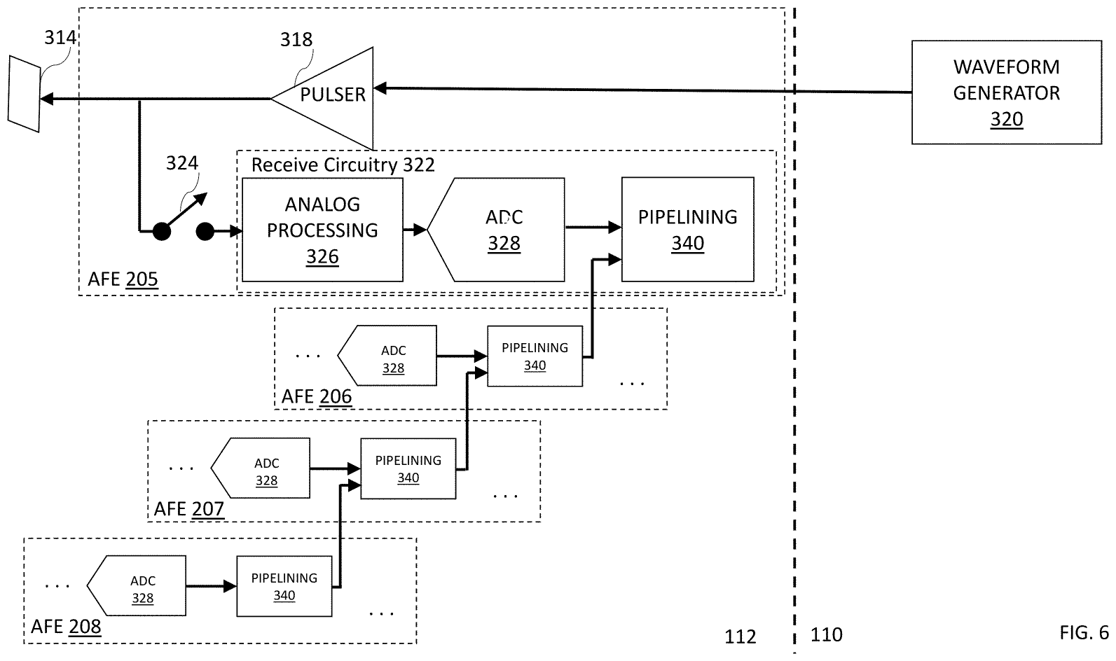

FIG. 6 illustrates another example block diagram of circuitry in the ultrasound-on-chip 100, in accordance with certain embodiments described herein. FIG. 6 differs from each of FIGS. 3-5 in that the embodiment of FIG. 6 lacks both the de-interleaving circuitry 342 and the digital processing circuitry 330. In this embodiment, the ultrasound-on-a-chip 100 may be configured to output the interleaved signals to an off-chip device, which may be configured to perform de-interleaving and digital processing, or which may be configured to digitally process interleaved signals.

It should be appreciated that the embodiments of FIGS. 3-6 are non-limiting, and the ultrasound-on-chip 100 may include fewer or more components than shown. For example, there may be additional components interposed between any components of the circuitry illustrated in FIGS. 3-6. However, even if there is (for example) more circuitry interposed between the ADC 328 and the pipelining circuitry 340, the ADC 328 may still be considered to “output” signals to the pipelining circuitry 340. Additionally, a first circuit component “coupled” to a second circuit component may mean that the first and second circuit components are directly coupled or that there is some other circuitry coupled between the first and second circuit components. In some embodiments, one waveform generator 320 may output to multiple pulsers 318 (e.g., in a multiplexed fashion). In some embodiments, one waveform generator 320 may output to only one pulser 318. In some embodiments, one pulser 318 may output to multiple ultrasonic transducers 314 (e.g., in a multiplexed fashion). In some embodiments, one pulser 318 may output to only one ultrasonic transducer 314. In some embodiments, multiple ultrasonic transducers 314 may output to one block of receive circuitry 322 (e.g., in a multiplexed fashion). In some embodiments, only one ultrasonic transducer 314 may output to one block of receive circuitry 322. In some embodiments, the ultrasonic transducer 314 may be configured to output to the ADC 328, and the analog processing circuitry 326 may be absent. In some embodiments, there may be multiple blocks of the digital processing circuitry 330, and each of the de-interleaved signals from the de-interleaving circuitry 342 may be outputted to a dedicated block of the digital processing circuitry 330. In some embodiments, there may be multiple blocks of the digital processing circuitry 330, and groups of the de-interleaved signals may each be multiplexed to one of the multiple blocks of the digital processing circuitry 330. In some embodiments, all the de-interleaved signals may be multiplexed to one block of the digital processing circuitry 330. In some embodiments, there may be multiple blocks of one type of digital processing circuitry (e.g., a dedicated block for each de-interleaved signal, or a block to which a group of de-interleaved signals is multiplexed) including certain circuitry, and then all the processed signals may be multiplexed to one block of a second type of digital processing circuitry. For example, the first type of digital processing circuitry may include one or more digital filters, digital beamforming circuitry, digital quadrature demodulation (DQDM) circuitry, averaging circuitry, digital dechirp circuitry, digital time delay circuitry, digital phase shifter circuitry, digital summing circuitry, and digital multiplying circuitry, and the second type of digital processing circuitry may include requantization circuitry, waveform removal circuitry, image formation circuitry, and backend processing circuitry.

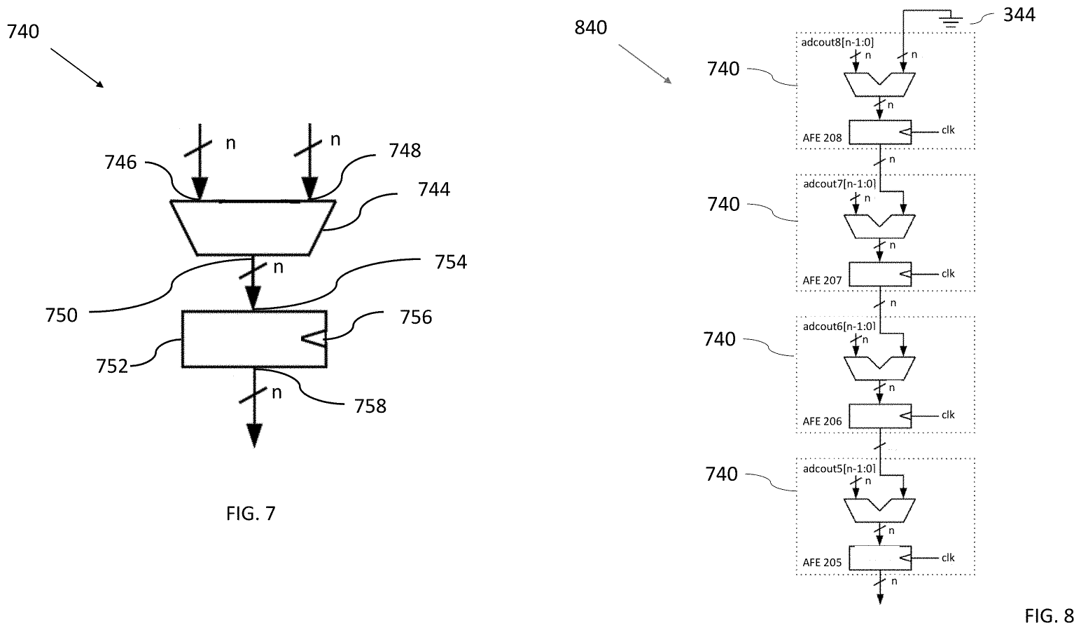

FIG. 7 illustrates example pipelining circuitry 740, in accordance with certain embodiments described herein. The pipelining circuitry 740 may be the pipelining circuitry 340 shown in any of the embodiments of FIGS. 3-6. The pipelining circuitry 740 includes a multiplexer 744 and a flip-flop 752. The multiplexer 744 includes a first data input 746, a second data input 748, and a data output 750. The flip-flop 752 includes a data input 754, a clock input 756, and a data output 758. The data output 750 of the multiplexer 744 is coupled to the data input 754 of the flip-flop 752. The multiplexer 744 may be configured to output either the signal from the first data input 746 or the signal from the second data input 748 at the data output 750. Internal circuitry in the multiplexer 744 (e.g., an internal counter) may control whether the multiplexer 744 outputs the signal from the first data input 746 or the signal from the second data input 748 at the data output 750. The flip-flop 752 may be a D-type flip-flop (DFF). The signals processed by the pipelining circuitry 740 are multi-bit signals. If the signal at the output 750 of the multiplexer 744 is configured to be n bits, the flip-flop 752 may include n flip-flops, each configured to take in as input and output one of the n bits. Collectively, the flip-flops constituting the flip-flop 752 may be configured, on a clock signal (e.g., a rising clock edge or a falling clock edge) at the clock input 756, to store the multi-bit signal on the data input 754 and provide that multi-bit signal at the data output 758.

FIG. 8 illustrates an example pipeline 840, in accordance with certain embodiments described herein. The pipeline 840 includes one block of the pipelining circuitry 740 for each of the AFEs 205-208. For simplicity, the first data input 746 of the multiplexer 744, the second data input 748 of the multiplexer 744, and the data output 758 of the flip-flop 752 will be referred to as the first input, the second input, and the output, respectively, of each block of pipelining circuitry 740. Also for simplicity, the output signal of the ADC 328 of the AFE 208 will be referred to as adcout8, the output signal of the ADC 328 of the AFE 207 will be referred to as adcout7, the output signal of the ADC 328 of the AFE 206 will be referred to as adcout6, and the output signal of the ADC 328 of the AFE 208 will be referred to as adcout8.

In the pipelining circuitry 740 of the AFE 208, the first input is coupled to adcout8, the second input is coupled to dummy data, namely 0 V (e.g., to ground 344), and the output is coupled to the second input of the pipelining circuitry 740 of the AFE 207. In the pipelining circuitry 740 of the AFE 207, the first input is coupled to adcout7, the second input is coupled to the output of the pipelining circuitry 740 of the AFE 208, and the output is coupled to the second input of the pipelining circuitry 740 of the AFE 206. In the pipelining circuitry 740 of the AFE 206, the first input is coupled to adcout6, the second input is coupled to the output of the pipelining circuitry 740 of the AFE 207, and the output is coupled to the second input of the pipelining circuitry 740 of the AFE 205. In the pipelining circuitry 740 of the AFE 205, the first input is coupled to adcout5, the second input is coupled to the output of the pipelining circuitry 740 of the AFE 206, and the output is the output of the pipeline 840 (and may be coupled, for example, to the de-interleaving circuitry 342). A clock signal clk is coupled to the clock input of the flip-flop 752 in each block of pipelining circuitry 740.

The pipeline 840 may be configured to operate as follows:

On clock cycle 1, each block of pipelining circuitry 740 may store and provide at its output the signal on its first input. Thus, the pipelining circuitry 740 of the AFE 205 may store and provide at its output adcout5 to the output of the pipeline 840, the pipelining circuitry 740 of the AFE 206 may store and provide at its output adcout6 to the second input of the pipelining circuitry 740 of the AFE 207, the pipelining circuitry 740 of the AFE 207 may store and provide at its output adcout7 to the second input of the pipelining circuitry 740 of the AFE 206, and the pipelining circuitry 740 of the AFE 208 may store and provide at its output adcout8 to the second input of the pipelining circuitry 740 of the AFE 207.

On clock cycle 2, each block of pipelining circuitry 740 may store and provide at its output the signal on its second input. Thus, the pipelining circuitry 740 of the AFE 205 may store and provide at its output adcout6 to the output of the pipeline 840, the pipelining circuitry 740 of the AFE 206 may store and provide at its output adcout7 to the second input of the pipelining circuitry 740 of the AFE 207, the pipelining circuitry 740 of the AFE 207 may store and provide at its output adcout8 to the second input of the pipelining circuitry 740 of the AFE 206, and the pipelining circuitry 740 of the AFE 208 may store and provide at its output 0 V to the second input of the pipelining circuitry 740 of the AFE 207.

On clock cycle 3, each block of pipelining circuitry 740 may store and provide at its output the signal on its second input. Thus, the pipelining circuitry 740 of the AFE 205 may store and provide at its output adcout7 to the output of the pipeline 840, the pipelining circuitry 740 of the AFE 206 may store and provide at its output adcout8 to the second input of the pipelining circuitry 740 of the AFE 207, the pipelining circuitry 740 of the AFE 207 may store and provide at its output 0 V to the second input of the pipelining circuitry 740 of the AFE 206, and the pipelining circuitry 740 of the AFE 208 may store and provide at its output 0 V to the second input of the pipelining circuitry 740 of the AFE 207.

On clock cycle 4, each block of pipelining circuitry 740 may store and provide at its output the signal on its second input. Thus, the pipelining circuitry 740 of the AFE 205 may store and provide at its output adcout8 to the output of the pipeline 840, the pipelining circuitry 740 of the AFE 206 may store and provide at its output 0 V to the second input of the pipelining circuitry 740 of the AFE 207, the pipelining circuitry 740 of the AFE 207 may store and provide at its output 0 V to the second input of the pipelining circuitry 740 of the AFE 206, and the pipelining circuitry 740 of the AFE 208 may store and provide at its output 0 V to the second input of the pipelining circuitry 740 of the AFE 207.

On clock cycle 5 or a subsequent clock cycle, when each of the ADCs 328 has produced a new value for adcout5, adcout6, adcout7, and adcout8, each block of pipelining circuitry 740 may store and provide at its output the signal on its first input, as described above with reference to clock cycle 1.

Thus, the signal that is outputted from the pipeline 840 may include adcout8, adcout7, adcout6, and adcout5 interleaved with each other. It should be appreciated that the pipeline 840 may help to reduce the number of wires passing over the AFEs 205-208. In particular, rather than a data bus from each AFE extending directly to the digital portion 110 of the UPU 200 which may, in an n-bit example, result in 4n wires (or, in embodiments with m AFEs in a column in a UPU, m×n wires) passing over the AFE 205 prior to reaching the digital portion 110), only n wires carrying the interleaved signals from the AFEs 205-208 may pass over any given AFE prior to reaching the digital portion 110 (as illustrated in FIG. 2). Reducing the number of wires passing over the AFEs 205-208 may reduce the effect of digital switching from data buses on analog circuitry in the AFEs 205-208. It should be appreciated that because four clock cycles may be required to output each of adcout8, adcout7, adcout6, and adcout5 from the pipeline 840, the clock rate of each of the ADCs 328 may be, at maximum, one-fourth (or, in embodiments with m AFEs in a column in a UPU, 1/m) the clock rate of the pipelining circuitry 740. It should also be appreciated that the 0 V signal that is inputted to the pipelining circuitry 740 of the AFE 208 may be interleaved with data from the ADC 328 of the AFE 208, data from the ADC 328 of the AFE 207, data from the ADC 328 of the AFE 206, and data from the ADC 328 of the AFE 205 at the output of the pipeline 840. The de-interleaving circuitry 342 may be configured to ignore this signal (i.e., not output it).

The pipeline 840 includes four blocks of pipelining circuitry 740 because there are 4 AFEs 205-208 in a column in an UPU 200, as illustrated in FIG. 2, whose data buses may need to pass over each other to reach the digital portion 110 of the UPU 200. It should be appreciated that there may be more or fewer blocks of pipelining circuitry 740 in the pipeline 840 if there are more or fewer AFEs in a column in the UPU 200.

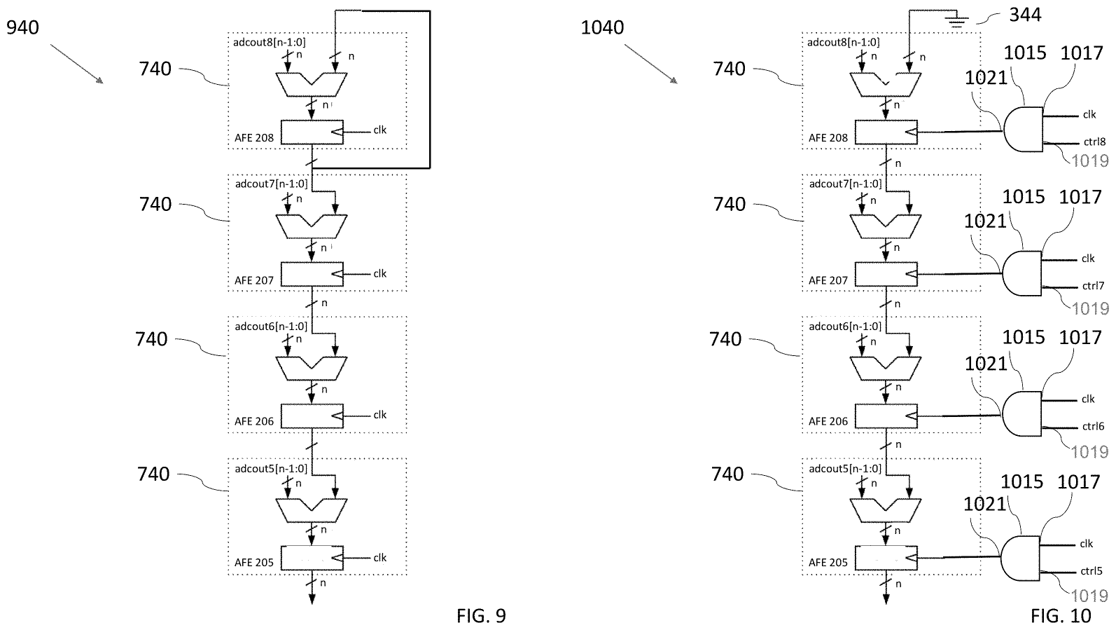

As described above, after a given block of pipelining circuitry 740 has shifted through adcout8, there is no further actual data for the block of pipelining circuitry 740 to shift, and subsequent shifting cycles are “dead” cycles for this block of pipelining circuitry 740. During these dead cycles, the block of pipelining circuitry 740 will shift through 0 V, which is originally inputted to the second input of the pipelining circuitry 740 of the AFE 208. This 0 V is dummy data. FIGS. 9-11 illustrate variations of the pipeline 840 to improve how dead cycles are handled.

FIG. 9 illustrates another example pipeline 940, in accordance with certain embodiments described herein. The pipeline 940 is the same as the pipeline 840, except that the second input of the pipelining circuitry 740 of the AFE 208 is coupled to the output of the pipelining circuitry 740 of the AFE 208, namely adcout8, rather than to 0 V. After storing adcout8 from the first input of the pipelining circuitry 740 of the AFE 208 and providing adcout8 at its output on clock cycle 1, on clock cycle 2, the pipelining circuitry 740 of the AFE 208 may store adcout8 again from the second input of the pipelining circuitry 740 of the AFE 208 and provide adcout8 at its output. Each subsequent block of pipelining circuitry 740 may then provide adcout8 at its output on consecutive clock cycles, rather than providing adcout8 on one clock cycle and providing 0 V on a subsequent clock cycle. This may be helpful, because the amount of digital switching on outputs of the blocks of pipelining circuitry 740 in transitioning from adcout8 to 0 V on consecutive block cycles may be greater than if adcout8 is outputted on consecutive clock cycles. Reducing digital switching may reduce draw in current from the power supply, power supply noise, and/or transfer of this digital switching activity through capacitive coupling to nearby high-precision, low bandwidth, and/or low amplitude analog signals, which in turn can reduce noise in measurements based on the analog signals. The second instance of adcout8 is dummy data, and it should be appreciated that in some embodiments, the second instance of adcout8 may not be outputted by the pipeline 940. Rather, before adcout8 is outputted for a second time by the pipeline 940, each block of pipelining circuitry 740 may store the signal at its first input, thus overwriting this second instance of adcout8.

FIG. 10 illustrates another example pipeline 1040, in accordance with certain embodiments described herein. The pipeline 1040 is the same as the pipeline 840, except that the clock input of each block of pipelining circuitry 740 may be gated by clock gating circuitry 1015. Each block of clock gating circuitry 1015 includes a first input 1017, a second input 1019, and an output 1021, and may be implemented using an AND gate, clock gating latch, glitchless clock-multiplexer, or other circuitry with similar function. The first input 1017 is coupled to the clocking signal clk and the second input 1019 is coupled to a control signal (ctrl5-ctrl8 for each of the AFEs 205-208, respectively). If the control signal is high, then clk will appear at the output 1021 of the clock gating circuitry 1015. If ctrl is low, then the output 1021 of the clock gating circuitry 1015 will be 0 V. In some embodiments, each of ctrl5-ctrl8 is high when its respective pipelining circuitry 740 is shifting useful data, namely data from ADCs. In other words, each of ctrol5-ctrl8 may be high until after their respective blocks of pipelining circuitry 740 have provided adcout8 at their output, at which point ctrl5-ctrl8 may be switched to low. In some embodiments, circuitry internal to the pipelining circuitry 740 that controls whether the multiplexer of the pipelining circuitry 740 of the AFE 208 selects data from the first input or the second input may also determine whether ctrl8 is high or low. When the pipelining circuitry 740 of the AFE 208 selects data from its first input (i.e., adcout8), ctrl8 may be high, and thus the clock gating circuitry 1015 may output clk to the clock input of the pipelining circuitry 740 the AFE 208. This may cause the pipelining circuitry 740 the AFE 208 to provide adcout8 at its output. However, when the pipelining circuitry 740 of the AFE 208 selects data from its second input (i.e., the 0 V dummy data), ctrl8 may be low, and thus the clock gating circuitry 1015 may output 0 V to the clock input of the pipelining circuitry 740 the AFE 208. This may prevent the pipelining circuitry 740 of the AFE 208 from updating its output with 0 V. Rather, the pipelining circuitry 740 of the AFE 208 may continue to provide adcout8 at its output. Thus, the amount of time when the pipelining circuitry 740 of the AFE 208 is being clocked may be reduced (e.g., by 75%) compared with when the pipelining circuitry 740 of the AFE 208 is continuously clocked (e.g., as in the pipeline 840). In this embodiment, ctrl5-ctrl7 may always be high. Thus, while the clock gating circuitry 1015 of AFEs 205-207 may not be used for clock gating, it may be simpler to replicate the all the blocks of pipelining circuitry 740 having the clock gating circuitry 1015 rather than instantiating certain blocks of pipelining circuitry 740 with clock gating circuitry 1015 and certain blocks without. In operation, as in the pipeline 940, after storing adcout8 from the first input of the pipelining circuitry 740 of the AFE 208 and providing adcout8 at its output on clock cycle 1, on clock cycle 2, the pipelining circuitry 740 of the AFE 208 may continue to provide adcout8 at its output. Each subsequent block of pipelining circuitry 740 after the AFE 208 may store and provide adcout8 at its output on consecutive clock cycles. It may be easier to realize this functionality with the clock gating circuitry 1015 of the pipeline 1040 rather than by routing adcout8 back to the second input of the pipelining circuitry 740 of the AFE 208 as in the pipeline 940.

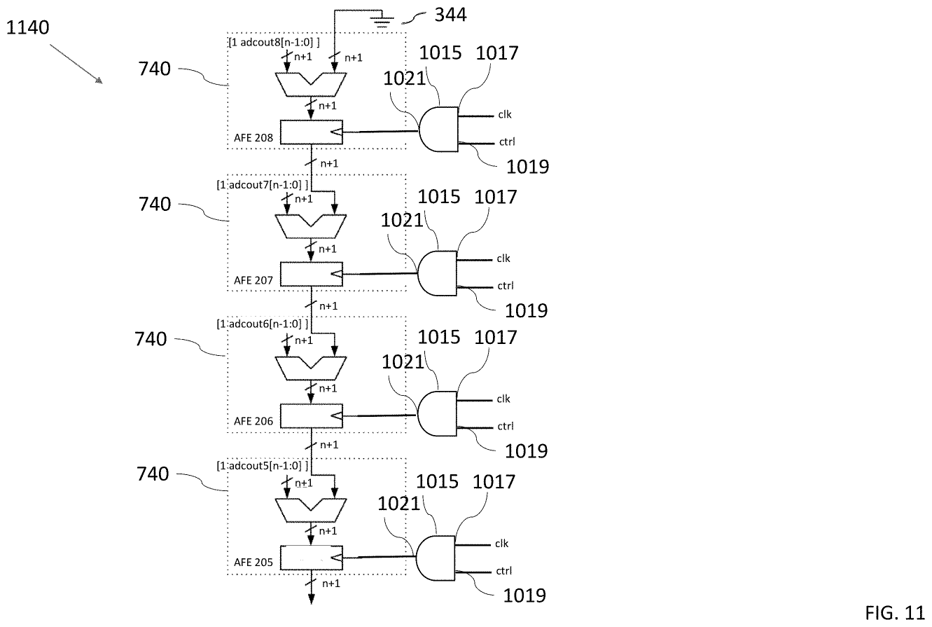

FIG. 11 illustrates another example pipeline 1140, in accordance with certain embodiments described herein. The pipeline 1140 is the same as the pipeline 1040, except that the n-bit signal from each ADC at the first input of each block of pipelining circuitry 740 is concatenated with a “valid” bit at the (n+1)^{th} bit position. The bit at the (n+1)^{th} position in the signal at the output of the multiplexer of each block of pipelining circuitry 740 may control each block of clock gating circuitry 1015. If the valid bit is 1, the clock gating circuitry 1015 may output clk from its first input, while if the valid bit is 0, the clock gating circuitry 1015 may output 0 V. Thus, if the multiplexer of a block of pipelining circuitry 740 has selected from its inputs ADC data, which has an accompanying valid bit of 1, the pipelining circuitry 740 may receive clk at its clock input from the clock gating circuitry 1015 and provide the ADC data at its output. Otherwise, if the block of pipelining circuitry 740 has selected 0 V (i.e., dummy data) from its inputs, which has an accompanying valid bit of 0, the pipelining circuitry 740 may receive 0 V from the clock gating circuitry 1015 at its clock input, and not provide the 0 V dummy data at its output. In short, the valid bit may cause all the blocks of pipelining circuitry 740 to shift through ADC data and to not shift through the 0 V dummy data. Thus, each block of pipelining circuitry 740 may only be clocked when necessary to shift through ADC data. This may be in contrast to certain embodiments of the pipeline 1040, in which only the clocking of the pipelining circuitry 740 of the AFE 208 may be reduced. However, the pipeline 1140 requires routing the extra valid bit.

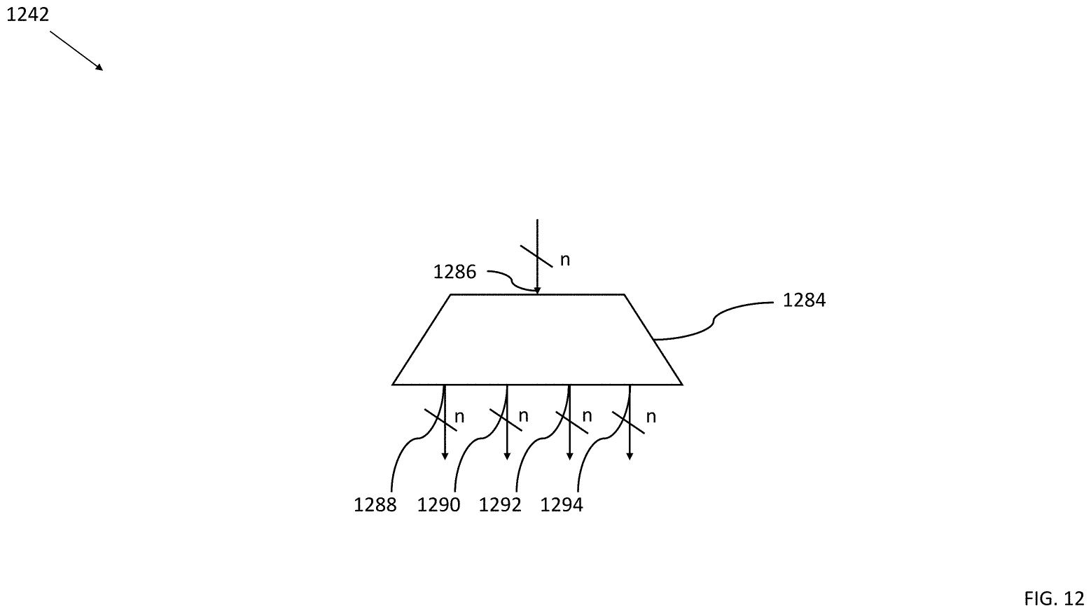

FIG. 12 illustrates example de-interleaving circuitry 1242, in accordance with certain embodiments described herein. The de-interleaving circuitry 1242 may be the de-interleaving circuitry 342 shown in the embodiments of FIG. 3 and/or FIG. 5. The de-interleaving circuitry 1242 includes a demultiplexer 1284. The demultiplexer 1284 includes a data input 1286, a first data output 1288, a second data output 1290, a third data output 1292, and a fourth data output 1294. The data input 1286 of the demultiplexer 1284 may be coupled to the output of a pipeline (e.g., the pipelines 840-1140). For example, the data input 1286 of the demultiplexer 1284 may be coupled to the data output of the pipelining circuitry 740 of the AFE 205. The demultiplexer 1284 may be configured to output the signal from the data input 1286 at the first data output 1288, the second data output 1290, the third data output 1292, or the fourth data output 1294. Internal circuitry in the demultiplexer 1284 (e.g., an internal counter) may control whether the demultiplexer 1284 outputs the signal from the data input 1286 at the first data output 1288, the second data output 1290, the third data output 1292, or the fourth data output 1294. For example, the signal at the data input 1286 of the demultiplexer 1284 (e.g., as received from a pipeline of pipelining circuitry) may be an interleaved signal including adcout5, adcout6, adcout7, adcout8, adcout5, adcout6, adcount7, adcout8, etc. The internal circuitry in the demultiplexer 1284 may be configured to cycle from outputting the signal at the data input 1286 at the first data output 1288, then the second data output 1290, then the third data output 1292, and then the fourth data output 1294. The demultiplexer 1284 may be configured to cycle through synchronized with the clock signal of the flip-flops in the pipelining circuitry 740. Thus, the demultiplexer 1284 may output adcout5 at the first data output 1288, adcout6 at the second data output 1290, adcout7 at the third data output 1292, and adcout8 at the fourth data output 1294. The signals processed by the de-interleaving circuitry 1242 are multi-bit signals.

The de-interleaving circuitry 1242 includes four outputs because there are 4 AFEs 205-208 in a column in an UPU 200, as illustrated in FIG. 2, whose signals may get interleaved with each other while passing in data buses over each other to reach the digital portion 110 of the UPU 200. It should be appreciated that there may be more or fewer outputs from the de-interleaving circuitry 1242 if there are more or fewer AFEs in a column in the UPU 200.

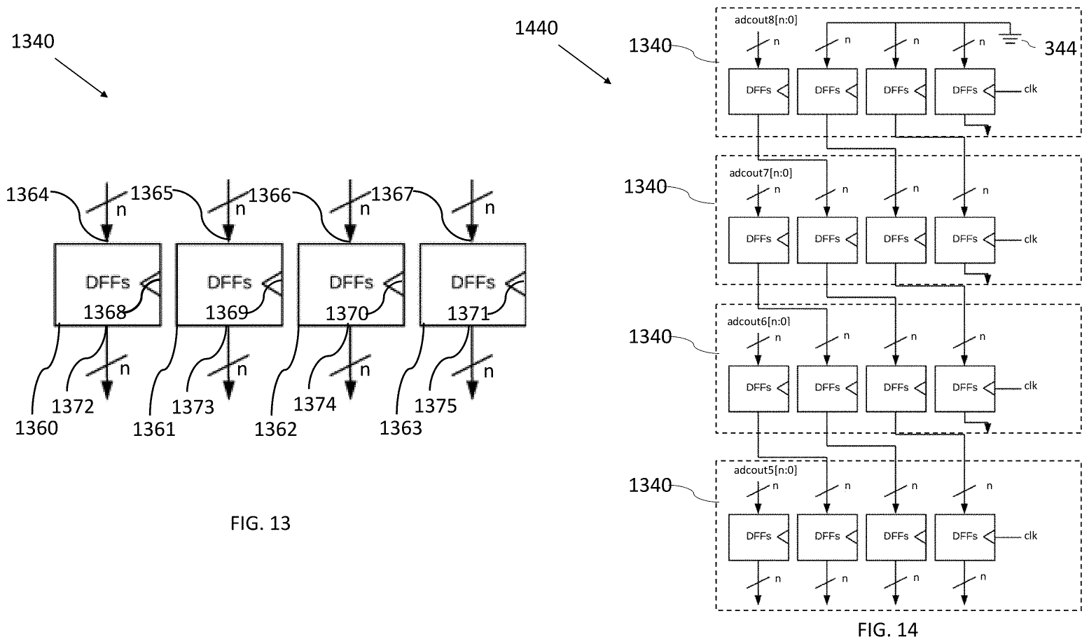

FIG. 13 illustrates another example of pipelining circuitry 1340, in accordance with certain embodiments described herein. The pipelining circuitry 1340 may be the pipelining circuitry 340 shown in any of the embodiments of FIGS. 3-6. The pipelining circuitry 1340 includes four flip-flops 1360-1363. The flip-flops 1360-1363 each have a data input 1364-1367, a clock input 1368-1371, and a data output 1372-1375, respectively. Further description of the flip-flops 1360-1363 may be found with reference to the flip-flop 752. The signals processed by the pipelining circuitry 1340 are multi-bit signals. The pipelining circuitry 1340 includes four flip-flops because there are 4 AFEs 205-208 in a column in an UPU 200, as illustrated in FIG. 2, whose data buses may need to pass over each other to reach the digital portion 110 of the UPU 200. It should be appreciated that there may be more or fewer flip-flops in the pipelining circuitry 1340 if there are more or fewer AFEs in a column in the UPU 200.

FIG. 14 illustrates an example pipeline 1440, in accordance with certain embodiments described herein. The pipeline 1440 one block of the pipelining circuitry 1340 for each of the AFEs 205-208. For simplicity, the data inputs 1364-1367 of the flip-flops 1360-1363 will be referred to as the first input, the second input, the third input, and the fourth input, respectively, of each block of pipelining circuitry 1340, and the data outputs 1372-1375 of the flip-flops 1360-1363 will be referred to as the first output, the second output, the third output, and the fourth output, respectively, of each block of pipelining circuitry 1340. Also for simplicity, the output signal of the ADC 328 of the AFE 208 will be referred to as adcout8, the output signal of the ADC 328 of the AFE 207 will be referred to as adcout7, the output signal of the ADC 328 of the AFE 206 will be referred to as adcout6, and the output signal of the ADC 328 of the AFE 208 will be referred to as adcout8.

In the pipelining circuitry 1340 of the AFE 208, the first input is coupled to adcout8 and the second, third, and fourth inputs are coupled to 0 V (e.g., to ground 344). The first output is coupled to the second input of the pipelining circuitry 740 of the AFE 207, the second output is coupled to the third input of the pipelining circuitry 740 of the AFE 207, the third output is coupled to the fourth input of the pipelining circuitry 740 of the AFE 207, and the fourth output is uncoupled. In the pipelining circuitry 1340 of the AFE 207, the first input is coupled to adcout7. The first output is coupled to the second input of the pipelining circuitry 740 of the AFE 206, the second output is coupled to the third input of the pipelining circuitry 740 of the AFE 206, the third output is coupled to the fourth input of the pipelining circuitry 740 of the AFE 206, and the fourth output is uncoupled. In the pipelining circuitry 1340 of the AFE 206, the first input is coupled to adcout6. The first output is coupled to the second input of the pipelining circuitry 740 of the AFE 205, the second output is coupled to the third input of the pipelining circuitry 740 of the AFE 205, the third output is coupled to the fourth input of the pipelining circuitry 740 of the AFE 205, and the fourth output is uncoupled. In the pipelining circuitry 1340 of the AFE 205, the first input is coupled to adcout5. The first output, the second output, the third output, and the fourth output are the outputs of the pipeline 1440. A clock signal clk is coupled to the clock input of each of the flip-flops 1360-1363 in each block of pipelining circuitry 1340 (although for simplicity, the connection of each flip-flip to clk is not illustrated).

In operation, the pipeline 1440 is configured to pass adcout8 from the first input of the pipelining circuitry 1340 of the AFE 208 through the pipeline 1440 and to the fourth output of the pipelining circuitry 1340 of the AFE 205, to pass adcout7 from the first input of the pipelining circuitry 1340 of the AFE 207 through the pipeline 1440 and to the third output of the pipelining circuitry 1340 of the AFE 205, to pass adcout6 from the first input of the pipelining circuitry 1340 of the AFE 207 through the pipeline 1440 and to the second output of the pipelining circuitry 1340 of the AFE 205, and to pass adcout5 from the first input of the pipelining circuitry 1340 of the AFE 205 through the pipeline 1440 and to the first output of the pipelining circuitry 1340 of the AFE 205. It should be appreciated that adcout8, adcout7, adcout6, and adcout5 at the output of the pipeline 1440 are not interleaved together, and thus the de-interleaving circuitry 342 may not be needed. For example, the ultrasound-on-chip 100 may use the circuitry of FIG. 6.

It should be appreciated that, in contrast to the pipelines 840-1140, the pipeline 1440 may not reduce the number of wires passing over any of the AFEs 205-208 prior to reaching the digital portion 110 of the UPU 200 compared with the case in which data buses extend directly from each of the AFEs 205-208 to the digital portion 110. However, because each data bus from a given AFE may only need to extend to an adjacent AFE (as illustrated in FIG. 2), the length of the data buses may be reduced from the case in which data buses extend directly from each of the AFEs 205-208 to the digital portion 110. This may reduce current draw from the power supply due to digital switching on the data buses (and in turn reduce the effect of digital switching on noise in the AFEs), since longer wires without registers may require larger buffers that may draw more current to meet timing requirements, as compared with shorter wires. Additionally, the pipeline 1440 may be able to transfer data at the system clock frequency, so the data rate may be as high as the system clock frequency.

It should be appreciated that the flip-flops in the pipeline 1440 that pass 0 V may not be necessary for pipelining of the ADC data. For example, only one flip-flop in the pipelining circuitry 1340 of the AFE 208 may be needed for pipelining the ADC data. However, all four flip-flops may be needed in the pipelining circuitry 1340 of the AFE 205. It may be simpler to replicate the pipelining circuitry 1340 having four flip-flops rather than instantiating pipelining circuitry 1340 having different numbers of flip-flops for each AFE. However, some embodiments may include pipelining circuitry 1340 having only the number of flip-flops needed for pipelining (i.e., no flip-flops passing 0 V).

The pipeline 1440 includes four blocks of pipelining circuitry 1340 because there are 4 AFEs 205-208 in a column in an UPU 200, as illustrated in FIG. 2, whose data buses may need to pass over each other to reach the digital portion 110 of the UPU 200. It should be appreciated that there may be more or fewer blocks of pipelining circuitry 1340 in the pipeline 1440 if there are more or fewer AFEs in a column in the UPU 200.

It should be appreciated that ultrasound transducers and any of the circuitry illustrated in FIGS. 3-14 may be integrated on a single semiconductor chip or on multiple semiconductor chips in a stacked configuration.

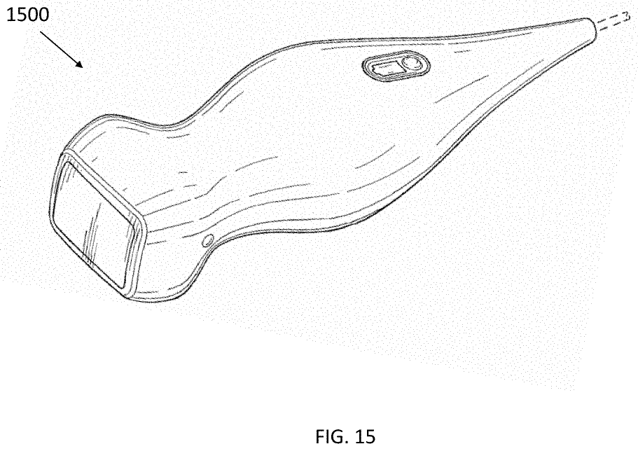

FIG. 15 illustrates an example handheld ultrasound probe 1500, in accordance with certain embodiments described herein. In some embodiments, an ultrasound-on-chip (e.g., the ultrasound-on-chip 100) including ultrasound transducers and any of the circuitry illustrated in FIGS. 3-14 may be integrated on this ultrasound-on-chip and disposed in the handheld ultrasound probe 1500.

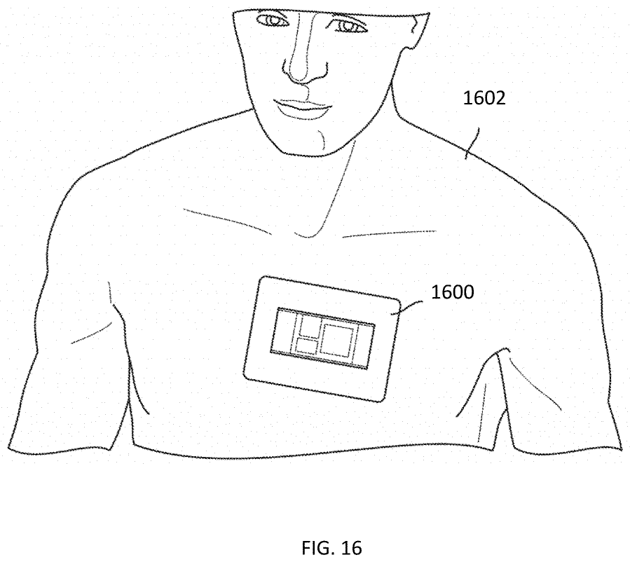

FIG. 16 illustrates an example wearable ultrasound patch 1600, in accordance with certain embodiments described herein. The wearable ultrasound patch 1600 is coupled to a subject 1602. In some embodiments, an ultrasound-on-chip (e.g., the ultrasound-on-chip 100) including ultrasound transducers and any of the circuitry illustrated in FIGS. 3-14 may be integrated on this ultrasound-on-chip and disposed in the wearable ultrasound patch 1600.

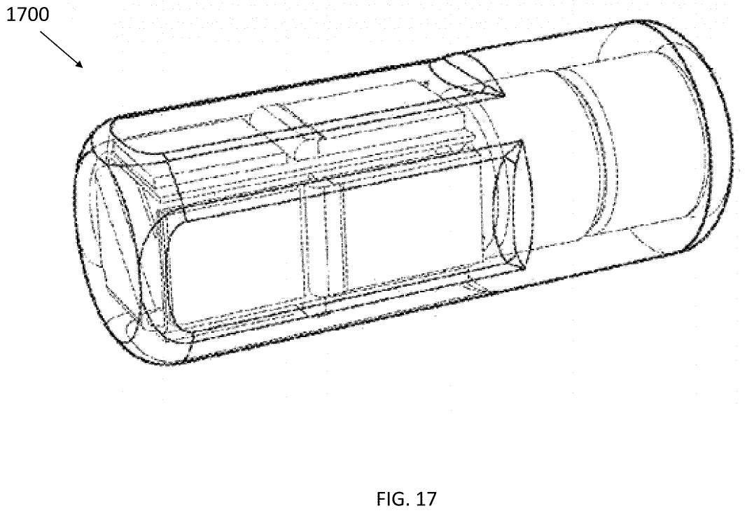

FIG. 17 illustrates an example ingestible ultrasound pill 1700, in accordance with certain embodiments described herein. In some embodiments, an ultrasound-on-chip (e.g., the ultrasound-on-chip 100) including ultrasound transducers and any of the circuitry illustrated in FIGS. 3-14 may be integrated on this ultrasound-on-chip and disposed in the ingestible ultrasound pill 1700.

Further description of the handheld ultrasound probe 1500, the wearable ultrasound patch 1600, and the ingestible ultrasound pill 1700 may be found in U.S. patent application Ser. No. 15/626,711 titled “UNIVERSAL ULTRASOUND IMAGING DEVICE AND RELATED APPARATUS AND METHODS,” filed on Jun. 19, 2017 and published as U.S. Pat. App. Publication No. 2017-0360399 A1 (and assigned to the assignee of the instant application).

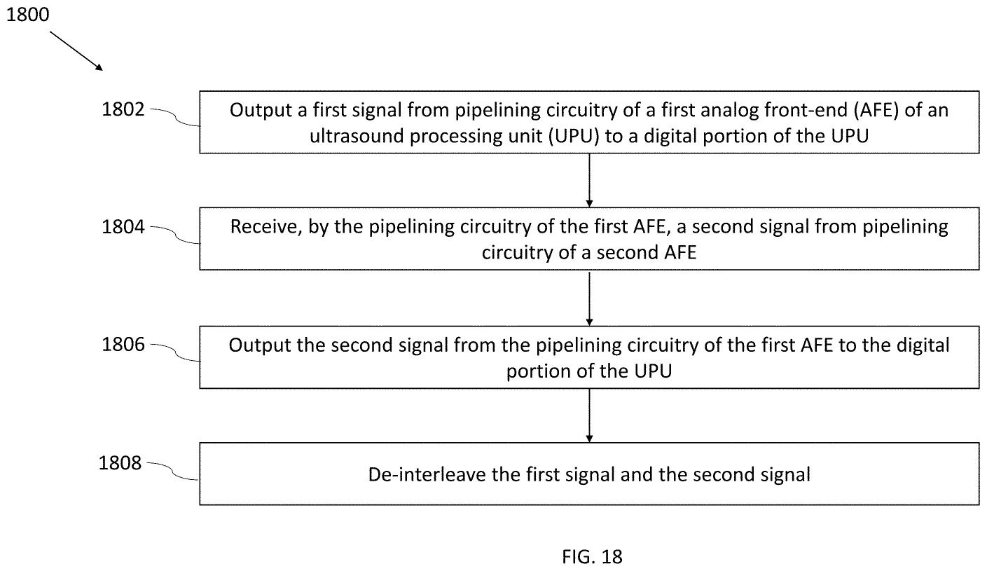

FIG. 18 illustrates a process 1800 for processing ultrasound signals, in accordance with certain embodiments described herein. The process 1800 is performed by an ultrasound processing unit (UPU) (e.g., a UPU 200). In some embodiments, the UPU may be in an ultrasound device such as the ultrasound-on-chip device 100. The ultrasound-on-chip device 100 may be, for example, in the handheld ultrasound probe 1500, the wearable ultrasound patch 1600, or the ingestible ultrasound pill 1700

Each UPU may be a self-contained ultrasound processing unit that forms a sub-array of a complete ultrasound imaging array in a scalable fashion. Each UPU may include an analog portion (e.g., the analog portion 112) and a digital portion (e.g., the analog portion 110. The analog portion may include multiple AFEs (e.g., the AFEs 201-208), each including a pulser (e.g., the pulser 318), a switch (e.g., the switch 324), analog processing circuitry (e.g., the analog processing circuitry 326), an ADC (e.g., the ADC 328), and pipelining circuitry (e.g., the pipelining circuitry 340, 740, and/or 1340). The digital portion may include de-interleaving circuitry (e.g., the de-interleaving circuitry 342 and/or 1242) and digital processing circuitry (e.g., the digital processing circuitry 330).

As described above, in some embodiments the waveform generator of a first AFE may be configured to provide a waveform to the pulser of the first AFE. The pulser may be configured to output a driving signal corresponding to the received waveform to an ultrasonic transducer (e.g., the ultrasonic transducer 314) of the first AFE. The ultrasonic transducer may be configured to emit pulsed ultrasonic signals into a subject, such as a patient, in response to the driving signal received from the pulser. The pulsed ultrasonic signals may be back-scattered from structures in the body, such as blood cells or muscular tissue, to produce echoes that return to the ultrasonic transducer. The ultrasonic transducer may be configured to convert these echoes into electrical signals. Analog processing circuitry of the first AFE may receive the electrical signals representing the received echoes from the ultrasonic transducer. The analog processing circuitry may include, for example, one or more analog amplifiers, one or more analog filters, analog beamforming circuitry, analog dechirp circuitry, analog quadrature demodulation (AQDM) circuitry, analog time delay circuitry, analog phase shifter circuitry, analog summing circuitry, analog time gain compensation circuitry, and/or analog averaging circuitry. The analog ultrasound signal output of the analog processing circuitry may be outputted to an ADC of the first AFE for conversion to a digital signal. The ADC may be, for example, a flash ADC, a successive approximation ADC, or a sigma-delta ADC configured to convert analog signals to digital signals. The digital ultrasound signal output of the ADC of the first AFE may be outputted to pipelining circuitry of the first AFE.

The pipelining circuitry may be configured to pipeline data from AFEs to the digital portion of the UPU. Generally, the pipelining includes a chain of AFEs passing their outputs (e.g., digital ultrasound signal output of their ADCs) from one AFE to another, until they reach the digital portion of the UPU. Acts 1802, 1804, and 1806 describe this pipelining from the perspective of pipelining circuitry of one AFE. Further description of pipelining circuitry may be found with reference to FIGS. 3-14.

In act 1802, the UPU outputs, from the pipelining circuitry of a first AFE, a first signal (e.g., the digital ultrasound signal output of the first AFE's ADC) to the digital portion of the UPU. The process 1800 proceeds from act 1802 to act 1804.

In act 1804, the pipelining circuitry of the first AFE of the UPU receives a second signal from pipelining circuitry of a second AFE. The process 1800 proceeds from act 1804 to act 1806.

In act 1806, the pipelining circuitry of the first AFE outputs the second signal (that was received from the pipelining circuitry of the second AFE) to the digital portion of the UPU. It should be appreciated that acts 1802 and 1804 may occur on one clock cycle (and may occur simultaneously), and act 1806 may occur on a second clock cycle. The process 1800 proceeds from act 1806 to act 1808.

It should be appreciated that the pipelining circuitry of the first AFE first outputs its own data and then outputs the data from the second AFE. In some embodiments, the first AFE may then output data from a third AFE, or from the first AFE again. In any case, the output to the digital portion of the UPU may be an interleaved signal including the data from the first AFE and the second AFE interleaved together.

In act 1808, the UPU de-interleaves the interleaved signal outputted by the pipelining circuitry of the first AFE. In particular, the UPU may split the interleaved signal into at least two separate signals, one for data from the first AFE and one for data from the second AFE. In some embodiments, act 1808 may be absent. For example, in certain embodiments (such as the embodiments of FIGS. 13-14), the pipelining circuitry may not output an interleaved signal, but rather output data from the first AFE at one output terminal and output data from the second AFE at another output terminal. Thus, de-interleaving may not be needed.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted, and one or more of the processes may include additional steps.

Various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An ultrasound processing unit (UPU), comprising:
a pipeline configured to pipeline ultrasound signals from multiple analog front-ends (AFEs) to a digital portion of the UPU.

2. The ultrasound processing unit of claim 1, wherein the ultrasound signals are digital ultrasound signals from analog-to-digital converters (ADCs) of the multiple AFEs.

3. The ultrasound processing unit of claim 1, wherein:
the pipeline comprises first pipelining circuitry in a first AFE of the multiple AFEs and second pipelining circuitry in a second AFE of the multiple AFEs; and
the first pipelining circuitry is configured to:
output a first digital ultrasound signal from the first pipelining circuitry to the digital portion of the UPU;
receive a second digital ultrasound signal from second pipelining circuitry; and
output the second digital ultrasound signal from the first pipelining circuitry to the digital portion of the UPU.

4. The ultrasound processing unit of claim 3, wherein the first pipelining circuitry is configured to output the first digital ultrasound signal and the second digital ultrasound signal to the digital portion of the UPU in an interleaved fashion.

5. The ultrasound processing unit of claim 4, further comprising:
de-interleaving circuitry coupled to the first pipelining circuitry and configured to de-interleave the first digital ultrasound signal and the second digital ultrasound signal outputted by the first pipelining circuitry.

6. The ultrasound processing unit of claim 5, wherein the digital portion comprises the de-interleaving circuitry.

7. The ultrasound processing unit of claim 5, wherein the de-interleaving circuitry comprises a demultiplexer comprising a first data input and multiple data outputs.

8. The ultrasound processing unit of claim 5, wherein:
the first pipelining circuitry comprises:
a multiplexer comprising a first data input, a second data input, and a data output; and
a flip-flop comprising a data input and a data output; wherein:
the data output of the multiplexer is coupled to the data input of the flip-flop; and
the de-interleaving circuitry comprises:
a demultiplexer comprising a data input and multiple data outputs; wherein:
the data output of the flip-flop of the first pipelining circuitry is coupled to the data input of the demultiplexer; and
the multiple data outputs of the demultiplexer are coupled to the digital portion of the UPU.

9. The ultrasound processing unit of claim 3, wherein:
the first AFE comprises an analog-to-digital converter (ADC) coupled to the first pipelining circuitry and configured to convert a first analog ultrasound signal to the first digital ultrasound signal;
the second AFE comprises an ADC coupled to the second pipelining circuitry and configured to convert a second analog ultrasound signal to the second digital ultrasound signal;
the UPU further comprises a data bus extending from the second pipelining circuitry to the first pipelining circuitry; and
the first pipelining circuitry is configured to receive the second digital ultrasound signal from the second pipelining circuitry over the data bus.

10. The ultrasound processing unit of claim 3, wherein:
the first AFE is disposed between the second AFE and the digital portion of the UPU.

11. The ultrasound processing unit of claim 3, wherein the first AFE further comprises a pulser, a switch, and analog processing circuitry.

12. The ultrasound processing unit of claim 3, wherein:
an ultrasound-on-chip comprises the UPU; and
the first and second AFEs are arranged along an elevational dimension of the ultrasound-on-chip.

13. The ultrasound processing unit of claim 12, further comprising:
ultrasonic transducers physically located on top of each of the first and second AFEs and arranged along the elevational dimension of the ultrasound-on-chip.

14. The ultrasound processing unit of claim 12, wherein the ultrasound-on-chip comprises an array of ultrasonic transducers along an azimuthal dimension and an elevational dimension of the ultrasound-on-chip.

15. The ultrasound processing unit of claim 3, wherein:
each of the first and second digital ultrasound signals comprises a number of bits; and
a number of wires carrying digital ultrasound signals from ADCs of the UPU and passing over the first AFE is equal to the number of bits.

16. The ultrasound processing unit of claim 3, wherein the pipelining circuitry of the first AFE is configured to:
output the first digital ultrasound signal from the first pipelining circuitry to the digital portion of the UPU and to receive the second digital ultrasound signal from the second pipelining circuitry on a first clock cycle; and
output the second digital ultrasound signal from the first pipelining circuitry to the digital portion of the UPU on a second clock cycle.

17. The ultrasound processing unit of claim 3, wherein each of the first pipelining circuitry and the second pipelining circuitry comprises:
a multiplexer comprising a first data input, a second data input, and a data output; and
a flip-flop comprising a data input and a data output; wherein:
the data output of the multiplexer is coupled to the data input of the flip-flop.

18. The ultrasound processing unit of claim 17, wherein:
the first data input of the multiplexer in the second pipelining circuitry is coupled to an ADC in the second AFE;
the data output of the flip-flop in the second pipelining circuitry is coupled to the second data input of the multiplexer of the first pipelining circuitry;
the first data input of the multiplexer in the first pipelining circuitry is coupled to an ADC in the first AFE; and
the data output of the flip-flop in the first pipelining circuitry is coupled to the digital portion of the UPU.

19. The ultrasound processing unit of claim 3, wherein:
the second pipelining circuitry comprises:
a flip-flop comprising a data input and a data output;
the first pipelining circuitry comprises:
a first flip-flop comprising a data input and a data output; and
a second flip-flop comprising a data input and a data output;
the data input of the flip-flop in the second pipelining circuitry is coupled to an ADC in the second AFE;
the data output of the flip-flop in the second pipelining circuitry is coupled to the data input of the second flip-flop in the first pipelining circuitry;
the data input of the first flip-flop in the first pipelining circuitry is coupled to an ADC in the first AFE; and
the data outputs of the first and second flip-flops in the first pipelining circuitry are coupled to the digital portion of the UPU.

20. The ultrasound processing unit of claim 1, wherein the digital portion comprises digital processing circuitry.
